(19) United States
(12) Patent Application Publication
(10) Pub. No.: US 2013/0279036 A1
(43) Pub. Date: Oct. 24, 2013

(54) ADJUSTABLE MIRROR ASSEMBLY ATTACHABLE TO THE LEG

(71) Applicant: Shamir Benita, D.N. Jezreel Valley (IL)

(72) Inventor: Shamir Benita, D.N. Jezreel Valley (IL)

(21) Appl. No.: 13/879,979

(22) PCT Filed: Oct. 11, 2011
(86) PCT No.: PCT/IL11/00812

§ 371 Date: Jul. 11, 2013

(30) Foreign Application Priority Data
Oct. 21, 2010 (IL) 208867

(51) Int. Cl. G02B 5/08

(52) U.S. Cl. CPC G02B 5/08
USPC 359/865

(57) ABSTRACT

An adjustable mirror device to be attached to a user's leg, for enabling a user to observe hidden intimate inspected areas, comprising (a) a holder straps to be wrapped around the leg of the user; (b) a mirror assembly that comprises one or more light reflecting mirroring surfaces; and (c) an adjustable arm that is fixedly attached in one end to the holder and in the other end, to the mirror assembly, for enabling the user to adjust each of the mirrors to be and remain in a variety of desired positions and orientations.

### FIELD OF THE INVENTION

The present invention relates to the field of human hygiene. More specifically, the present invention is related to a device for easily observing intimate areas of the human body, particularly, the groin area.

### BACKGROUND OF THE INVENTION

One of the issues of feminine (or more generally, human) hygiene is related with taking care of the pubic hair. For this necessity and many others, a woman or a man who wants to observe normally hidden areas without the need for assistance of other person should have a comfortable view of her\his intimate organs.

There are several prior art documents that disclose mirrors for overcoming observation problems but all of them are insufficient for fulfilling the above specific need.

U.S. Pat. No. 5,311,366 to discloses a mirror adapted for use in self-catheterizing procedures which is adjustable to a plurality of inclinations and enables the person to view the body cavity while leaving the persons hands free. However, this disclosed mirror device is movable only around one axis and using it forces the person to sit in a particular position which might be uncomfortable.

U.S. Pat. No. 6,273,575 discloses a self-inspection apparatus which includes a frame that supports a mirror. The frame is placeable along the front edge of a toilet bowl and is secured thereto by a toilet seat for holding said mirror in front of the exterior surface of the toilet bowl in a position facing the toilet bowl. However, this device is not easy to store and it can not be easily carried. Moreover, using it is restricted only to a toilet room.

U.S. Pat. No. 4,257,680 discloses a mirror assembly for patients with personal hygiene problems. This mirror assembly is compact but it is not adjustable and therefore, it may cause inconvenience to the user, since instead of being able to move the mirror she (or he) has to move the body.

It is therefore an object of the present invention to provide a portable mirror device for observing hidden intimate organs and the organs themselves.

Another object of the present invention is to provide a mirror device for observing hidden intimate organs, which is easy to use and not restricted to a toilet room.

It is still an object of the present invention to provide a mirror device for observing hidden intimate organs which is portable compact adjustable and independent of the presence of a toilet seat or any other device.

It is yet an object of the present invention to provide a mirror device for observing hidden intimate organs which is of low cost and easy to manufacture.

Other objects advantages of the present invention will become clear as the description proceeds.

### SUMMARY OF THE INVENTION

The present invention is directed to an adjustable mirror device to be attached to a user's leg, for enabling a user to observe hidden intimate inspected areas, that comprises:
(a) A holder, having one or more straps to be wrapped around the leg of the user;
(b) A mirror assembly that comprises one or more light reflecting mirroring surfaces; and
(c) an adjustable arm being fixedly attached in one end to the holder and in the other end, to the mirror assembly, for enabling the user to adjust each of the mirrors to be and remain in a variety of desired positions and orientations.

The strap may be secured to the user's leg by a Velcro or by a buckle.

The adjustable member may be connected to the mirror by a spherical joint. The mirror may be protected by in a foldable case.

The mirror device may further comprise a light source powered by a battery and a switch, for enabling better inspection of the hidden inspected areas.

At least one light reflecting mirroring surfaces may be a curved and magnifying surface.

### BRIEF DESCRIPTION OF THE DRAWINGS

The above and other characteristics and advantages of the invention will be better understood through the following illustrative and non-limitative detailed description of preferred embodiments thereof, with reference to the appended drawings, wherein:

FIG. 1 illustrates a general view of a mirror device, proposed by the present invention;

FIG. 2 is an exemplary implementation of a mirror device, according to an embodiment of the present invention;

FIG. 3 is a perspective view of the mirror assembly, according to an embodiment of the present invention; and

FIG. 4 illustrates the back side of the mirror assembly, according to an embodiment of the present invention.

### DETAILED DESCRIPTION OF THE INVENTION

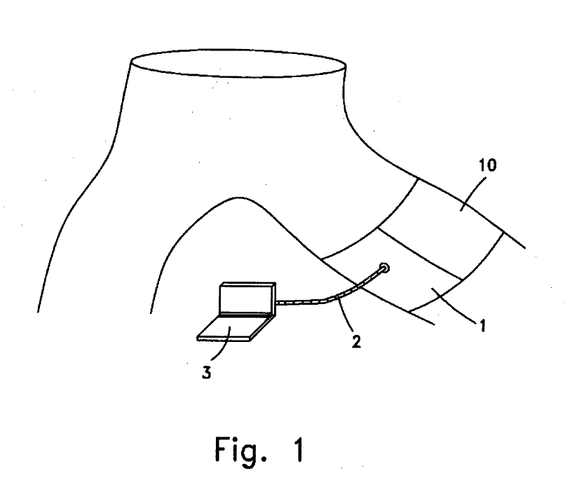

FIG. 1 illustrates a general view of a mirror device 10 proposed by the present invention, which is attached to the upper portion of user's leg by an appropriate holder that functions as an essentially stationary base when the leg does not move. An adjustable arm is fixedly connected to the holder by its proximal end and carries the mirror by its distal end.

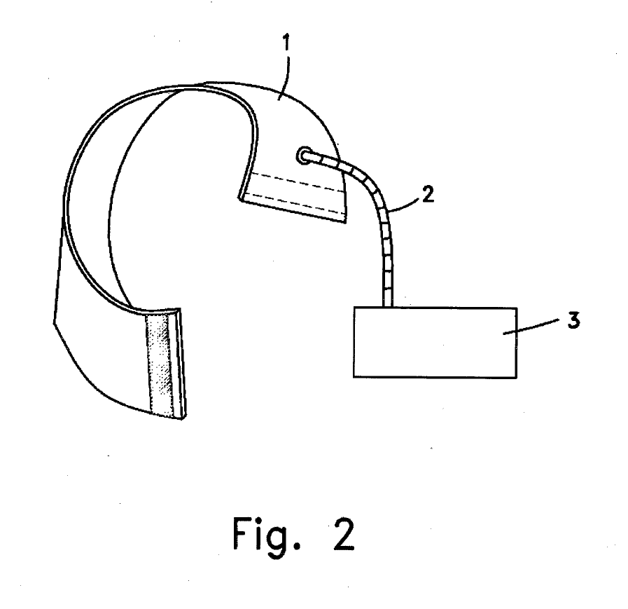

As illustrated in FIG. 2, the present invention discloses an exemplary implementation of a mirror device 10, which is attachable to the user's leg and includes the following parts:
(a) a strap 1 which is adapted to be wrapped and buckled around the user's leg.
(b) An adjustable holding arm 2, fixedly connected to strap 1 in one end and to mirror 3 at the other end, for enabling the user to adjust the orientation of mirror 3, so to be in a variety of desired positions.
(c) A mirror assembly 3, which may include a flat mirror with a desired area that allows the user to observe a sufficient inspected area. Alternatively, mirror assembly 3 may further include an additional curved mirror 3a, for allowing the user to focus on a particular (and smaller) area of interest and obtain a magnified observation of this particular area.

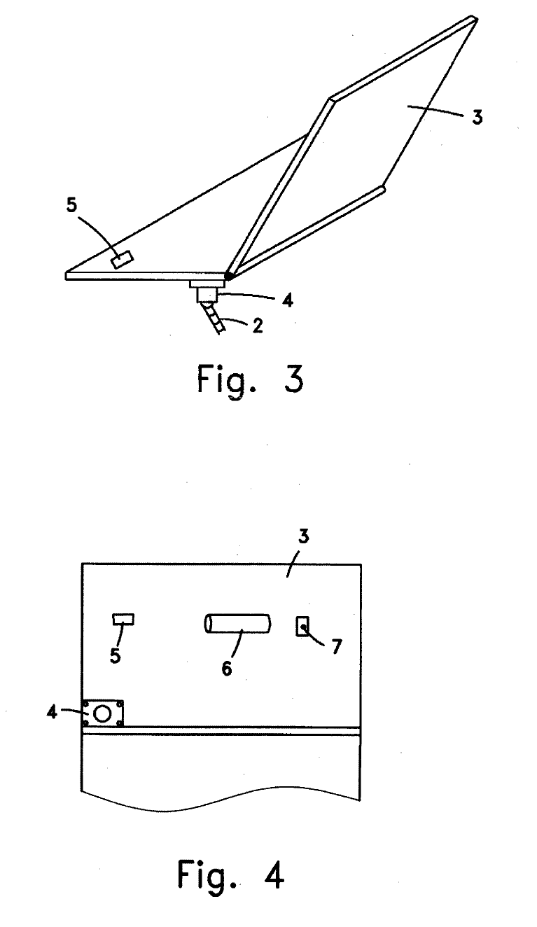

FIG. 3 is a perspective view of the mirror assembly, according to an embodiment of the invention. The mirror assembly 3 is a foldable mirror, to which a spherical joint 4 is attached. In this configuration, the spherical joint 4 connects the adjustable arm 2 to one of the foldable parts of the mirror assembly 3. One foldable part may include a flat mirror, while that other foldable part may include a curved magnifying mirror. A light source 5, such as a LED, may be attached to one of the sections of the foldable mirror 3, for providing a clearer view of the area of interest.

FIG. 4 illustrates the back side of the mirror assembly 3, according to an embodiment of the invention. The spherical joint 4 is attached to the back side of one section of the foldable mirror 3. The battery 6 is attached to the back side of a section of the foldable mirror assembly 3 and provides power supply to the light source 5, which may be mounted on the sidewall of the mirror reflecting surface. The user may switch the light on and off by a mechanical switch 7 that may be mounted on the backside, as well.

The flexible arm 2 may be connected to the mirror 3 via a ball joint.

The holding strap may be secured to the leg by a buckle or by a bonding surface, such as Velcro.

In order to protect the fragile mirrors, the mirror assembly 3 may be enclosed in a protecting foldable case.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. An adjustable mirror device to be attached to a user's leg, for enabling a user to observe hidden intimate inspected areas, comprising:
(a) A holder, having one or more straps to be wrapped around the leg of said user;
(b) A mirror assembly that comprises one or more light reflecting mirroring surfaces; and
(c) an adjustable arm being fixedly attached in one end to said holder and in the other end, to said mirror assembly, for enabling said user to adjust each of said mirrors to be and remain in a variety of desired positions and orientations.

2. An adjustable mirror device according to claim 1, wherein the strap is secured to the user's leg by a Velcro or by a buckle.

3. An adjustable mirror device according to claim 1, wherein the adjustable member is connected to the mirror by a spherical joint.

4. An adjustable mirror device according to claim 1, wherein the mirror is protected by in a foldable case.

5. An adjustable mirror device according to claim 1, further comprising a light source powered by a battery and a switch, for enabling better inspection of hidden inspected areas.

6. An adjustable mirror device according to claim 1, wherein at least one light reflecting mirroring surfaces is a curved and magnifying surface.

7. An adjustable mirror device according to claim 2, wherein the adjustable member is connected to the mirror by a spherical joint.
